(19) United States
(12) Patent Application Publication
(10) Pub. No.: US 2019/0365281 A1
(43) Pub. Date: Dec. 5, 2019

(54) PHYSIOLOGIC MONITORING DECISION SUPPORT SYSTEM COMBINING CAPNOMETRY AND OXYGEN SATURATION

(71) Applicant: KONINKLIJKE PHILIPS N.V., EINDHOVEN (NL)

(72) Inventors: JOSEPH ALLEN ORR, PARK CITY, UT (US); LARA MARIE BREWER, BOUNTIFUL, UT (US); PAUL BRUCE GUNNESON, CHESHIRE, CT (US)

(21) Appl. No.: 16/478,046

(22) PCT Filed: Jan. 15, 2018
(86) PCT No.: PCT/EP2018/050799

§ 371 Date: Jul. 15, 2019

Related U.S. Application Data
(60) Provisional application No. 62/446,608, filed on Jan. 16, 2017.

(51) Int. Cl. A61B 5/083; A61B 5/00; A61B 5/1455

(52) U.S. Cl. CPC A61B 5/0836; A61B 5/14551; A61B 5/746

(57) ABSTRACT

A patient monitoring device includes a capnograph device (10) and a pulse oximeter (70). An electronic processor (84) is programmed to generate a capnography index (50) indicative of patient well-being from a capnogram measured by the capnograph device, and to generate an arterial blood oxygen saturation (SpO2) index (90) indicative of patient well-being from SpO2 (72) measured by the pulse oximeter. A patient safety index (92) is computed from the capnography index and the SpO2 index. One or more clinical warnings are determined based at least in part on the patient safety index. A display component (82) is configured to display at least one of the computed one or more clinical warnings.

### FIELD

The following relates generally to the capnography arts, medical monitoring arts, and related arts.

### BACKGROUND

A capnography device monitors the concentration or partial pressure of carbon dioxide (CO₂) in respiratory gases. Capnography is commonly used in conjunction with mechanically ventilated patients in order to assess respiratory system status. A skilled anesthesiologist can usually evaluate the capnogram (that is, the CO₂ trend line as measured by a capnograph device) to assess respiratory health.

Capnography is increasingly used as a more generic vital sign for assessing patient health. For example, capnography may be used to monitor a patient who is breathing spontaneously and not undergoing mechanical ventilation, using a side stream capnograph device configuration in which respired air is sampled via a nasal cannula in conjunction with a dedicated sampling pump. In these broader contexts, medical personnel with limited expertise in anesthesiology are required to assess respiratory health on the basis of capnograph data. To facilitate this, it is common for the capnograph device to be programmed to output standard derived parameters, particularly respiration rate (RR) and end-tidal CO₂ (etCO₂). The RR is the breathing rate, quantified as the (quasi-)periodicity of the capnogram waveform. The etCO₂ is the partial pressure at the end of the exhalation phase. However, since the expired CO₂ is usually highest at the end of the exhalation phase, etCO₂ is commonly defined as the maximum observed CO₂ partial pressure over the breathing cycle.

While RR and etCO₂ are useful parameters, they do not capture the rich informational content of the capnogram waveform. To this end, it is also known to perform automated capnogram waveform analyses, designed to mimic clinical analyses that might be performed by a skilled anesthesiologist. For example, Colman et al., U.S. Pat. No. 8,412,655 and Colman et al., U.S. Pat. No. 8,414,488 disclose capnogram waveform analyses such as correlating pauses with apnea events, correlating a long downward slope of the capnogram waveform with possible partial airway obstruction, correlating a low capnogram waveform with possible low cardiac output, correlating a rounded capnogram waveform with a possible problem with the nasal cannula, or so forth. Based on such waveform analyses, the capnograph device may provide informational messages such as “open airway”, “check airway”, “possible low cardiac output”, “check cannula interface”, or so forth.

Capnogram waveform analyses provide richer information from the capnogram, but entail complex processing such as detecting the breath cycling, amplitude and period normalization, and segmenting regions of the capnogram waveform within each breath cycle. These complex analyses introduce numerous possible error mechanisms such as incorrect waveform segmentation or information loss during the normalization operations.

Some additional background references include the following.

WO 2016/108121 A1 published Jul. 7, 2016 discloses, among other aspects, a gas concentration monitoring system that may include a processor configured to detect a concentration of a selected gas in a sample gas flow obtained from a physical interface to a patient. A dataset is formed, including a plurality of data points, each data point corresponding to the detected concentration of the selected gas within the sample gas flow during a sampling time. The data set may be variously employed. For example, the data points may be grouped according to a frequency of occurrence of the data points within the sampling time. A signal confidence and/or signal quality may be determined based on relative characteristics between the groups of data points. WO 2016/108121 A1 claims priority to U.S. Ser. No. 62/098,367 filed Dec. 31, 2014. WO 2016/108121 A1 and U.S. Ser. No. 62/098,367 are each incorporated herein by reference in its entirety.

WO 2016/108127 A1 published Jul. 7, 2016 discloses, among other aspects, a capnography system. A controller is configured to obtain a sample gas flow from a physical interface for a patient. A change is determined in a characteristic of the sample gas flow during a sampling time interval. It is determined whether the change in the characteristic of the sample gas flow during the sampling time interval is equal to or greater than a corresponding threshold value. It is determined that supplemental oxygen is provided when it is determined that the change in the characteristic of the sample gas flow is equal to or greater than the threshold value. It is determined that supplemental oxygen is not provided when it is determined that the change in the characteristic of the sample gas flow is less than the threshold value. WO 2016/108127 A1 claims priority to U.S. Ser. No. 62/097,946 filed Dec. 30, 2014. WO 2016/108127 A1 and U.S. Ser. No. 62/097,946 are each incorporated herein by reference in its entirety.

U.S. Ser. No. 62/203,416 titled “Capnography with Decision Support System Architecture” filed Aug. 11, 2015 is incorporated herein by reference in its entirety. U.S. Ser. No. 62/203,416 discloses, among other aspects, a capnograph device that includes a carbon dioxide measurement component and an electronic processor programmed to generate a capnogram comprising carbon dioxide level sample values measured as a function of time. End-tidal carbon dioxide (etCO₂) is determined from the capnogram, and an etCO₂ parameter quality index (etCO₂ PQI) is computed using one or more quantitative capnogram waveform metrics computed from the capnogram. A respiration rate (RR) value is also determined from the capnogram, and a RR PQI is computed using the RR value and the etCO₂ PQI. A respiratory well-being index (RWI) may be computed from the etCO₂ and RR values and the etCO₂ and RR PQI values. In some embodiments the one or more capnogram waveform metrics are computed from a capnogram histogram generated from the capnogram.

The following discloses a new and improved systems and methods that address the above referenced issues, and others.

### SUMMARY

In one disclosed aspect, a patient monitoring device comprises a capnograph device, a pulse oximeter, and an electronic processor programmed to: generate a capnography index indicative of patient well-being from a capnogram measured by the capnograph device; generate an arterial blood oxygen saturation (SpO₂) index indicative of patient well-being from SpO₂ measured by the pulse oximeter; compute a patient safety index from the capnography index and the SpO₂ index; and compute one or more clinical warnings determined based at least in part on the patient safety index. A display component may be configured to display at least one of the computed one or more clinical warnings.

In another disclosed aspect, a non-transitory storage medium stores instructions readable and executable by an electronic processor to perform patient monitoring comprising: generating a capnography index indicative of patient well-being from a capnogram measured by a capnograph device; generating an arterial blood oxygen saturation (SpO₂) index indicative of patient well-being from SpO₂ (72) measured by a pulse oximeter; and computing a patient safety index from the capnography index and the SpO₂ index.

One advantage resides in providing a capnograph device whose output more effectively assesses patient respiratory health.

Another advantage resides in providing a capnograph device outputting derived parameters characterizing the detailed capnogram waveform without requiring breath detection or segmentation of the capnogram waveform.

Another advantage resides in more accurate respiratory system status information from capnogram data.

Another advantage resides in providing clinical decision support that synergistically combines capnography information with pulse oximetry information.

Another advantage resides in providing clinical decision support employing both capnography information with pulse oximetry information, which provides a ranked list of clinical warnings generated by each constituent monitoring modality.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a capnograph device.

FIG. 2 diagrammatically illustrates an idealized capnogram and the corresponding capnogram histogram.

FIG. 3 diagrammatically plots the CO₂ fall time for an idealized capnogram waveform (top plot) and a capnogram with supplemental oxygen washout (bottom plot).

FIG. 4 diagrammatically illustrates a patient monitor that provides a Patient Safety Index (PSI) generated from a respiratory well-being index (RWI) generated by the capnography device of FIG. 1 and a blood oxygenation level (e.g. SpO₂) generated by a pulse oximeter.

FIGS. 5 and 6 illustrate examples of an SpO₂ index-versus-SpO₂ value function suitably used in the patient monitor of FIG. 4, for the case of no supplemental oxygen (FIG. 5) and for the case of supplemental oxygen (FIG. 6).

### DETAILED DESCRIPTION

In some embodiments disclosed herein, parameter quality indices are computed to quantitatively assess the reliability of the respiratory rate (RR) and end-tidal CO₂ (etCO₂) evaluated from the capnogram. A respiratory well-being index (RWI) may also be computed, based in part on the etCO₂ parameter quality index (etCO₂ PQI) and the RR parameter quality index (RR PQI). These parameter quality indices enable medical personnel to interpret the capnogram using conventional tools, especially the RR and etCO₂, but provide metrics (the quality control indices) to assist medical personnel in assessing whether the RR and etCO₂ are reliable data for making clinical decisions.

Further, in some embodiments the parameter quality indices are computed at least in part using a histogram of CO₂ value counts versus (binned) CO₂ level. This histogram is computed over a time interval encompassing several breaths. For example, the histogram is acquired over a 30 second time interval in one illustrative embodiment, which corresponds to about 6-10 breaths for a normal adult patient respiration interval of 3-5 seconds/breath (12-20 breaths per minute), up to 30 breaths for a rapidly respiring infant (respiration rate of 60 breaths per minute).

Advantageously, the capnogram histogram is computed without segmenting the waveform into different regions (e.g. inspiration, expiration) and without segmenting individual breath cycles (that is, without a breath detector). The capnogram histogram advantageously has a “standard” shape for a normally respiring patient, due to the typical capnogram pattern in which the CO₂ level is close to zero during the inspiration phase and close to its maximum (i.e. close to etCO₂ for the patient) during the expiration phase. These two phases define respective low and high regions of the disclosed capnogram histogram, with a third transitional histogram region in-between. Rich information about the capnogram waveform can be extracted from the capnogram histogram, without reliance upon the difficult and often imprecise task of segmenting the capnogram waveform into breath cycles which are then further segmented into inspiration and expiration time intervals.

In particular, the etCO₂ parameter quality index (PQI) is computed primarily or entirely using the histogram. In some embodiments the etCO₂ PQI is computed further based on capnogram characteristics that can be quantified without segmenting the capnogram into inspiration and expiration regions. Illustrative embodiments of the etCO₂ PQI do rely upon breath detection and capnogram waveform segmentation, as the RR is intimately associated with (indeed defined by) the breath cycle. However, the RR PQI is optionally further based on the etCO₂ PQI thereby incorporating waveform information from the capnogram histogram.

The RWI is computed based on the etCO₂ and RR values, and further based on the etCO₂ PQI and RR PQI. Incorporating the PQI values into the RWI captures the recognition herein that a poor capnogram waveform is often an indication of poor respiratory health, rather than being an indicator of a capnograph measurement problem.

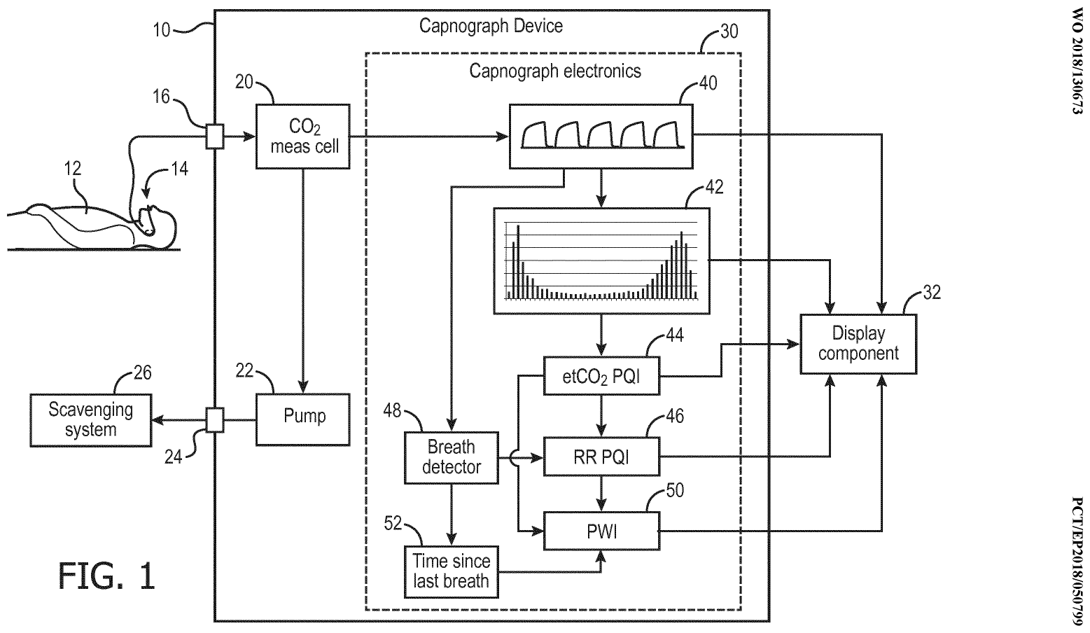

With reference to FIG. 1, an illustrative capnograph device 10 is connected with a patient 12 by a suitable patient accessory, such as a nasal cannula 14 in the illustrative example, or by an airway adaptor or so forth. The patient accessory 14 may optionally include one or more ancillary components, such as an air filter, water trap, or the like (not shown). In the illustrative capnograph 10, respired air is drawn from the patient accessory 14 into a capnograph air inlet 16 and through a carbon dioxide (CO₂) measurement component or cell 20 by an air pump 22. The air is then discharged via an air outlet 24 of the capnograph 10 to atmosphere or, as in the illustrative embodiment, is discharged through the air outlet 24 into a scavenging system 26 to remove an inhaled anesthetic or other inhaled medicinal agent before discharge into the atmosphere. The CO₂ measurement component or cell 20 may, for example comprise an infrared optical absorption cell in which carbon dioxide in the respired air drawn from the patient accessory 14 produces absorption that is detected by an infrared light source/detector assembly.

The illustrative capnograph device 10 has a sidestream configuration in which respired air is drawn into the capnograph device 10 using the pump 22, and the CO₂ measurement cell 20 is located inside the capnograph device 10. That is, the sidestream capnograph device 10 includes, as a unit, the carbon dioxide measurement component 20, the electronic processor 30, and the pump 22 connected to draw respired air though the carbon dioxide measurement component 20. The sidestream configuration is suitably used for a spontaneously breathing patient, i.e. a patient who is breathing on his or her own without assistance of a mechanical ventilator. In an alternative configuration, known as a mainstream configuration (not illustrated), the CO₂ measurement cell is located externally from the capnograph device housing, typically as a CO₂ measurement cell patient accessory that is inserted into the “mainstream” airway flow of the patient. Such a mainstream configuration may, for example, be employed in conjunction with a mechanically ventilated patient in which the CO₂ measurement cell patient accessory is designed to mate into an accessory receptacle of the ventilator unit, or is installed on an airway hose feeding into the ventilator. The disclosed approaches for quantitatively assessing parameter quality and patient respiratory well-being are readily applied either in conjunction with a sidestream capnograph device (as in the illustrative example of FIG. 1) or in conjunction with a mainstream capnograph device.

With continuing reference to FIG. 1, the capnograph device 10 (in either the illustrative sidestream configuration or in the alternative mainstream configuration) includes capnograph electronics 30 which provide power and control for operating the CO₂ measurement cell 20 and (in the sidestream configuration) the pump 22. Note that the power and control links are not illustrated in diagrammatic FIG. 1. The capnograph electronics 30 additionally perform processing of the CO₂ signal output by the CO₂ measurement cell 20, as diagrammatically indicated in FIG. 1 and as described herein. Clinical data output by the capnograph 10 are displayed on a display component 32, stored in an electronic medical record (EMR) or the like, or otherwise utilized. The display component 32 may be a component of the capnograph or, as illustrated in FIG. 1, the display component 32 may be an external display component connected to the capnograph 10. For example, the external display component 32 may be a multi-function bedside patient monitor and/or a nurses' station patient monitor or so forth. It will be further appreciated that the capnograph may include numerous other components not illustrated in simplified diagrammatic FIG. 1, such as a pressure gauge, flow meter, and so forth.

The capnograph electronics 30 may be variously implemented, such as by a suitably programmed electronic processor, e.g. a microprocessor or microcontroller of the capnograph 10. While a single electronics unit 30 is illustrated, it is alternatively contemplated to employ various combinations of electronics, for example different electronic components may be operatively interconnected to implement a pump, power supply, infrared light source and detector, power supply (for the CO₂ measurement cell 20), analog-to-digital conversion circuitry (to sample the infrared light detector of the CO₂ measurement cell 20), and so forth. Still further, it is contemplated for the electronics that perform the capnograph data processing to be disposed outside of the capnograph device itself. For example, the capnograph data processing may be performed by electronics in another device (for example, the computer of a nurses' station that receives the CO₂ signal from the measurement cell 20, or that receives a capnogram generated by the capnograph device and performs further processing). It will be still further appreciated that the capnograph data processing disclosed herein as being performed by the capnograph electronics 30 may be embodied by a non-transitory storage medium storing instructions that are readable and executable by the microprocessor, microcontroller, or other electronic processor to perform the disclosed capnograph data processing. Such non-transitory storage media may, by way of non-limiting illustration, include a hard disk drive or other magnetic storage medium, a flash memory, read-only memory (ROM) or other electronic storage medium, an optical disk or other optical storage medium, various combinations thereof, or so forth.

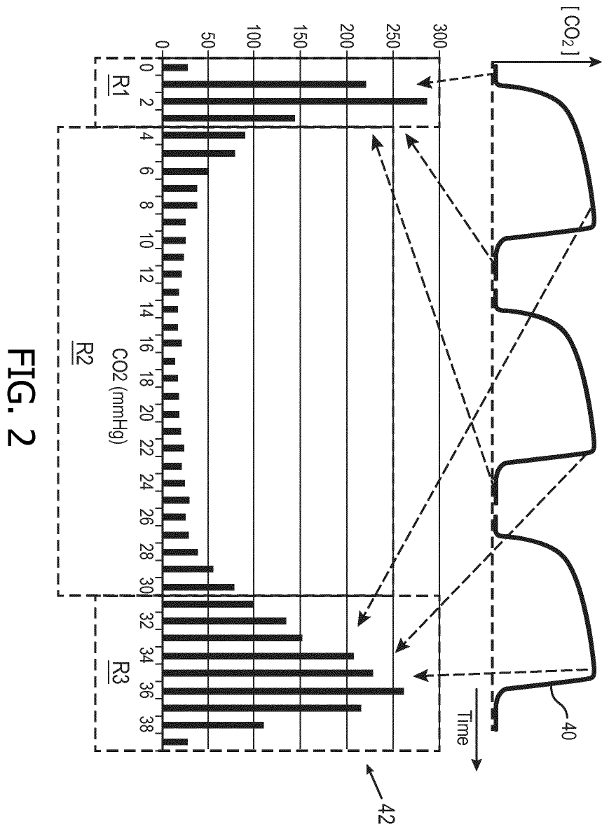

With continuing reference to FIG. 1 and with further reference to FIG. 2, an illustrative embodiment of the capnograph data processing performed by the capnograph electronics 30 (or alternatively in whole or in part by a nurses' station monitor, bedside patient monitor, or other device with a suitably programmed electronic data processor) is diagrammatically shown in FIG. 1. The CO₂ signal is sampled and optionally corrected for factors such as the presence of interfering gases (e.g. nitrous oxide), barometric pressure, and so forth in order to generate a capnogram 40. The capnogram 40 is a signal representing the partial pressure or concentration of carbon dioxide, denoted in FIG. 2 as [CO₂], as a function of time. Diagrammatic FIG. 2 illustrates the capnogram 40 as an idealized waveform for a healthy patient, in which every breath is identical and exhibits near-zero [CO₂] during the inspiratory phase and a well-defined maximum [CO₂] that rises gradually over the expiratory phase and terminates in a maximum [CO₂] corresponding to end-tidal CO₂, and in which the etCO₂ is the same for every breath. In practice, it will be understood that the capnogram 40 for a real patient usually deviates significantly from this idealized curve due to numerous factors such as non-uniform breathing, talking, coughing, possible chronic lung problems in the case of an ill patient, or so forth. In the capnogram of a real patient, the etCO₂ may vary from breath to breath. The illustrative idealized example of FIG. 2 further assumes a constant respiration rate. Again, in a real patient, the RR is generally not constant—the RR can increase significantly due to excitement or exertion, may slow during rest periods, may stop entirely during a sleep apnea episode, and/or may generally vary significantly due to various respiratory ailments or other medical conditions.

With continuing reference to FIGS. 1 and 2, the capnograph electronics 30 are programmed to compute a capnogram histogram 42 from the capnogram 40. The capnogram histogram 42 is a histogram of CO₂ sample values (y-axis) versus CO₂ level (x-axis). The capnogram histogram 42 is computed for a sliding window of duration 30 seconds (for illustrative FIG. 2; other window sizes are contemplated, preferably of a duration long enough to encompass several breaths). By way of illustrative example, if the CO₂ measurement cell 20 acquires samples at 10 msec intervals (100 samples per second) and the window is 30 seconds then, for each capnogram sample in the 30 second window (consisting of 3000 points) the bin corresponding to the CO₂ value for the point is incremented. In the capnogram histogram of a typical capnogram, there is a lower baseline region during inspiration and an elevated CO₂ region during expiration. Between these two regions, there are a set of points that make up the rising and falling edges of the capnogram. More particularly, as delineated in FIG. 2, three regions R1, R2, R3 can be defined. Region R1 of the histogram 42 includes the points in the capnogram 40 measured by the CO₂ measurement cell 20 during the inspiratory phase of the breath. In the illustrative example of FIG. 2, Region R1 includes the bins from 0 to 3 mmHg. Region R2 of the histogram 42 includes all the points from the capnogram 40 forming the rising and falling edges in the capnogram 40. In the illustrative example of FIG. 2, Region R2 includes the bins from 4 to 30 mmHg. Finally, Region R3 of the histogram 42 includes the points in the capnogram 40 measured during the expiratory phase of the breath. In the illustrative example of FIG. 2, Region R3 includes all the bins from 31 to 39 mmHg.

The capnogram histogram of a typical capnogram has certain characteristics. The histogram for a typical capnogram will have a higher number of occurrences of CO₂ sample values in the bins of Region R1 and Region R3, and the number of occurrences in the bins of Region R2 should be lower than the number of occurrences in Regions R1 and R3. That is, the capnogram histogram 42 has a peak in lower Region R1 and a peak in upper Region R3, and a valley in the intermediate Region R2. Further, the peak in upper Region R3 is typically more spread-out than the peak in Region R1, as seen in the idealized capnogram histogram 42 of FIG. 2. The spread in the peak in upper Region R3 is caused by the slope of the capnogram 40 during the exhalation phase, with the highest CO₂ value typically occurring at the end of the breath (i.e. end-tidal point). This slope of the capnogram waveform 40 is reflected in spreading of the points making up the peak in upper Region R3 of the capnogram histogram 42. Such spreading may additionally or alternatively be caused by the usual situation in which every breath does not have the same peak CO₂ value (or, said another way, etCO₂ varies from breath to breath). The difference in etCO₂ value for each breath is reflected in spreading of the peak in upper Region R3. By contrast, during the inspiration phase of the capnogram the CO₂ level usually falls to a flat baseline level that is close to zero, and exhibits little variation from breath to breath, leading to a narrower peak in the lower Region R1 of the histogram 42.

The capnogram histogram 42 is computed from the capnogram 40 in the sliding window, with a new histogram computed every few seconds, e.g. every 5 seconds in one illustrative example employing a 30 second window. There is no attempt to synchronize the window with an integer number of breaths, but the window is preferably large enough to encompass several breaths (e.g. for a normal adult patient respiration interval of 3-5 seconds/breath the illustrative 30 sec window encompasses 6-10 breaths). By re-computing the histogram on a shorter time interval than the window size (e.g. every 5 sec using a 30 sec window) the successive histogram windows significantly overlap providing for a smoothing effect as a function of time. Since there is no synchronization with the breath cycling, there is no need to employ a breath detector in constructing the capnogram histogram 42, and the determination of the histogram 42 is a very fast CO₂ sample binning process.

An end-tidal carbon dioxide (etCO₂) value and a respiratory rate (RR) value are determined from the capnogram signal 40. Substantially any technique to detect a signal maximum can be used to detect the etCO₂ value. For example, in some embodiments, the etCO₂ value is determined from the capnogram signal 40 by analysis of the histogram 42 derived from the capnogram signal 40. In this approach, the CO₂ level of the highest CO₂ level bin having a non-zero sample count provides an etCO₂ value. Similarly, substantially any technique to determine periodicity of a signal can be used to detect the RR value. For example, the RR value can be determined by detecting breaths using the breath detector 48 and thereby determining breath intervals (the RR being the inverse of the average breath interval). Alternatively, a Fast Fourier Transform (FFT) can be applied to determine the RR value in the frequency domain.

With continuing reference to FIG. 1, the capnogram histogram 42 is used to compute an end-tidal CO₂ parameter quality index (etCO₂ PQI) 44. This index is computed as a weighted sum of parameters derived from the capnogram histogram 42, and optionally also from the capnogram 40 itself. The parameters included in the weighted sum are suitably chosen as relevant criteria in determining the confidence in the etCO₂ measurement obtained from the capnogram 40. In one illustrative embodiment, the etCO₂ PQI 44 is computed from parameters including (1) a metric of the portion of the histogram 42 that is above the baseline; (2) a metric of the difference between the maximum CO₂ in Region R3 and the CO₂ level in Region R3 having the highest histogram counts; (3) a metric comparing the Region R3 count versus the Region R2 count; (4) a metric of the fraction of the total counts in Region R3; and (5) a metric of the CO₂ fall time.

The metric of the portion of the histogram 42 that is above the baseline characterizes the portion of the histogram that is in Region R3 as compared with Region R1. This metric is large for a normal capnogram, but may be low in the case of a poor capnogram waveform having an inconsistent expiratory plateau.

The metric of the difference between the maximum CO₂ in Region R3 and the CO₂ level in Region R3 having the highest histogram counts is expected to be small because the end-tidal point should have the largest CO₂ value and a CO₂ level bin at or close to etCO₂ should also have a large number of counts since the expiratory plateau usually flattens as it approaches the end-tidal point. This metric may be computed from the difference between the CO₂ level of the bin of Region R3 having a non-zero count and the CO₂ level of the bin of Region R3 storing the highest count.

The metric comparing the upper Region R3 count versus the intermediate Region R2 count quantifies the expectation that a sharp transition should be present from the inspiratory phase to the expiratory phase in the capnogram 40. In such a case, the intermediate Region R2 count is low and the upper Region R3 count is high. However, since there are more bins in intermediate Region R2 than in upper Region R3, this metric may preferably be quantified using the average count over all bins of Region R2, and likewise using the average count over all bins of Region R3.

The metric of the fraction of the total counts in upper Region R3 should be high since a large portion of the capnogram waveform consists of the expiratory phase. This metric may be computed using the ratio of the total counts in upper Region R3 to the total counts in the capnogram histogram 42.

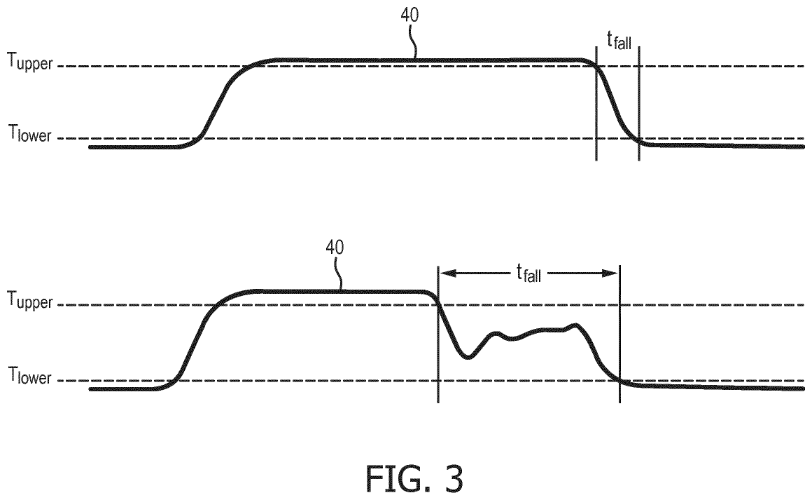

With brief reference to FIG. 3, the metric of the CO₂ fall time differs from the previous four metrics contributing to the illustrative etCO₂ PQI in that the metric of the CO₂ fall time is computed from the capnogram 40 rather than from the capnogram histogram 42. The metric of the CO₂ fall time is useful for detecting when the capnogram waveform is washed out due to the effect of supplemental oxygen. This is illustrated in FIG. 3. The top plot of FIG. 3 shows the expiratory plateau for the same idealized capnogram 40 as is shown in FIG. 2. The CO₂ fall time is computed as the time interval from when a high CO₂ level falls below an upper threshold Tᵤₚₚₑᵣ until the CO₂ level decreases below a lower threshold T_{lower}. This CO₂ fall time is indicated as t_{fall} in the top plot of FIG. 3 showing the idealized capnogram 40. It is seen that t_{fall} is relatively short. By contrast, FIG. 3 bottom plot shows a capnogram 40_{O2} which exhibits supplemental oxygen washout. In this case, the transition from Tᵤₚₚₑᵣ to T_{lower} is much longer.

It will be noted that the CO₂ fall time can be determined without performing breath detection, and without segmenting the capnogram waveform into inspiratory and expiratory phases. For example, in the illustrative example the CO₂ fall time is computed by identifying when a high CO₂ level falls below Tᵤₚₚₑᵣ and then when it falls below T_{lower}.

With returning reference to FIG. 1, the etCO₂ PQI 44 is suitably computed as a weighted sum of these metrics (and/or other metric that correlate with the reliability of the etCO₂ measurement by capnography). That is:

$${et CO}_{2} PQI = {\sum }_{i} {W}_{i} * {S}_{i}$$

where the index i ranges over the metrics contributing to etCO₂ PQI 44, Sᵢ is the score (i.e. value) of the ith metric, and Wᵢ is a weight for the ith metric. The weights may be generated manually (e.g. based on assessment by a skilled pulmonologist, anesthesiologist, respiratory therapist or other expert of the relative importance of the various metrics) or by performing machine learning using a training set of representative capnograms each labeled by a skilled pulmonologist, anesthesiologist, or other expert as to reliability of the etCO₂ value obtained from the training capnogram.

The five metrics contributing etCO₂ PQI in the example are merely illustrative. More generally, it will be appreciated that the capnogram histogram 42 is expected to exhibit a large narrow peak in a lower Region R1 corresponding to the inspiration phase of the respiratory cycle, a large slightly broader peak in an upper Region R3 corresponding to the expiratory phase, and a deep valley in an intermediate Region R2 corresponding to transitions from inspiration-to-expiration and from expiration-to-inspiration. Deviations from this basic histogram shape are expected when the capnogram waveform is degraded, and consequently etCO₂ values are expected to be less reliable. Various metrics can be constructed and optimized using histograms constructed for training capnograms in order to quantitatively characterize metrics to assess the histogram shape and hence the capnogram waveform. The optimal choice of metrics, and their weights, depends on the capnograph device and its connection to the patient, the demographic being monitored, the desired sensitivity (e.g. how “bad” should the capnogram waveform be before the etCO₂ PQI starts to significantly decrease), and so forth. In some embodiments the metrics may be optimized for different patient connections (e.g. nasal cannula versus airway adaptor), different patient breathing conditions (e.g. spontaneous breathing versus various mechanical ventilation modes), or so forth. The capnogram histogram shape reflects the capnogram waveform, so quantitative metrics of the histogram provide assessment of the capnogram waveform quality without the need to detect breath intervals in the capnogram and without the need to segment the capnogram into inspiration and expiration phases. In the illustrative example, one metric (CO₂ fall time) is extracted directly from the capnogram 40 rather than from the capnogram histogram 42, but this is still done without performing breath detection or segmenting the capnogram into breathing phases. The computations are fast, and can be performed in real-time (that is, with a delay of a few tens of seconds, a few seconds, or less).

With continuing reference to FIG. 1, a respiratory rate parameter quality index (RR PQI) 46 is also determined. The RR, and the RR PQI, both depend on detecting breaths, and hence receive as input the breath intervals detected in the capnogram 40 by a breath detector 48. The RR PQI 46 is suitably determined as a weighted sum of metrics including, by way of illustrative example: respiratory rate (RR), a metric of expiration time/inspiration time ratio (IE ratio), a metric quantifying invalid peak counts in the capnogram of a breath, a capnogram carbon dioxide level dynamic range metric, and a metric of how close the inspiratory CO₂ level is to zero. The RR and IE ratio values should be in reasonable ranges (e.g. RR around 12-20 breaths per minute for an adult) so values falling significantly outside the reasonable reduce the RR PQI 46. Extra (invalid) peaks can result in erroneous breath detections, hence more invalid peaks reduces the RR PQI. The capnogram dynamic range (maximum CO₂ level minus minimum CO₂ level) impacts the signal strength, so a low dynamic range reduces RR PQI. Similarly, the CO₂ level should be close to zero during inspiration; whereas, a higher CO₂ level during inspiration makes breath detection more difficult leading to a lower value for the RR PQI 46.

In the illustrative embodiment of FIG. 1, the RR PQI 46 is also determined based on the etCO₂ PQI 44 which serves as an additional metric in the weighted sum. The etCO₂ PQI 44 is a metric of “normalcy” of the capnogram waveform. Since a highly abnormal capnogram waveform makes breath detection more difficult, a lower value for the etCO₂ PQI 44 results in a lower RR PQI value as well. Employing the etCO₂ PQI 44 as an input metric to the RR PQI 46 advantageously re-uses the etCO₂ PQI 44 in assessing reliability of the RR.

The RR PQI 46 is again suitably computed as a weighted sum of the contributing metrics:

$$RRPQI = {\sum }_{i} {W}_{i} * {S}_{i}$$

where the index i ranges over the metrics contributing to RR PQI 46, Sᵢ is the score (i.e. value) of the ith metric, and Wᵢ is a weight for the ith metric. The weights again may be generated manually or by performing machine learning using a training set of representative capnograms labeled as to RR reliability. The metrics contributing RR PQI in the example are again merely illustrative, and additional or other metrics are contemplated.

In some embodiments, a respiratory well-being index (RWI) 50 is also computed, which represents a quality score to assess the respiratory well-being of a patient using the capnogram 40. The RWI 50 is designed to help medical personnel evaluate the overall respiratory well-being of the patient. RWI 50 may also be used to identify non-intubated patients who are at risk for hypoventilation due to central or obstructive apnea, such as during procedural sedation. In a suitable embodiment, metrics that serve as weighted inputs to the RWI 50 include the measured RR and etCO₂ and the corresponding RR PQI 44 and etCO₂ PQI 46. In general, if either the RR or etCO₂ are outside their respective normal ranges then this lowers the RWI 50. A lower RR PQI 44 or lower etCO₂ PQI 46 also lowers the RWI 50. In some embodiments, a time-since-last-breath metric is also incorporated into the RWI 50 in order to facilitate its use in detecting airway obstruction or apnea episodes. For example, the time-since-last-breath may be quantified from the capnogram 40 by a block 52 assessing the time since last elevated CO₂ level.

The indices 44, 46, 50 are suitably re-calculated each time the capnogram histogram 42 is updated, e.g. every 5 seconds in the illustrative example. Since the illustrative histogram calculation window is 30 seconds, the first calculation of the indices 44, 46, 50 is performed after 30 seconds of the capnogram 40 are acquired.

If the capnograph device 10 is programmed to provide informational messages based on the RR and etCO₂ values, then the indices 44, 46, 50 may optionally be used to suppress these informational messages when the underlying RR or etCO₂ is unreliable as indicated by the corresponding PQI. By way of non-limiting illustration, in one contemplated embodiment the messaging scheme of Table 1 is employed, with the outputs only being displayed when RWI is lower than some threshold value.

TABLE 1

| | Message when the | Message when the |
|---|---|---|
| | parameter is above | parameter is below |
| Parameter | the “Normal” range | the “Normal” range |
| etCO₂ | “Hypo-ventilation” | “Shallow breaths” “try |
| | | chin lift” |
| etCO₂ PQI Score | N/A | “Shallow breaths” “try |
| | | chin lift” |
| RR | “Patient anxious” | “Low resp rate”, |
| | (do not show message | “prompt patient to |
| | if RR PQI is low) | breathe” |
| RR PQI score | N/A | “Unstable breathing” |
| Time since high CO₂ | “Long time since | N/A |
| signal | breath”, “prompt patient | |
| | to breathe” | |

In this illustrative messaging scheme, the “patient anxious” message is suppressed if the RR PQI 46 is below a threshold value.

In addition to (or in place of) computing and displaying (on the display component 32) values of probative parameters such as etCO₂, RR, etCO₂ PQI 44, RR PQI 46, and/or RWI 50, it is contemplated to display on the display component 32 the capnogram histogram 42 itself. As previously discussed, the capnogram histogram 42 embodies substantial information about the capnogram waveform in a format that may be more readily perceived by medical personnel as compared with reading a display of the capnogram 40 (which may optionally also be displayed on the display 32, e.g. as a trend line). One advantage of displaying the capnogram histogram 42 as compared with displaying a trend line of the capnogram 40 is that the trend line is typically scrolled horizontally, whereas the capnogram histogram 42 does not scroll and is updated, e.g. every 5 seconds with substantial overlap between successive updates due to the large window overlap between successive updates (e.g. with a 30 second window and 5 sec updates, each successive histogram is derived from 25 seconds of the same capnogram data that was used to generate the immediately previous histogram and only 5 seconds of new capnogram data).

The foregoing embodiments advantageously provide capnography monitoring with output that is more readily comprehended and acted upon by medical personnel. In some embodiments which follow, the capnography monitoring is synergistically combined with blood hemoglobin oxygen saturation information, for example arterial blood oxygen saturation (SpO₂) measured by a pulse oximeter which measures the pulsatile part of blood in the finger or other tissue at which the SpO₂ measurement is made. While venous blood is the majority of the blood in the finger, venous blood does not pulse significantly, and hence is not considered in the SpO₂ measurement. Only the arterial blood pulsates strongly, and hence the pulse oximeter measures the arterial blood oxygen saturation. The term “arterial” refers to blood that has not yet participated in gas exchange (causing loss of O₂ captured in the lungs and collection of CO₂ from the tissues). It may be noted that the arterial blood may be located in arteries or in capillaries (including small capillaries)—such blood is nonetheless arterial blood even if located in the capillaries, so long as it has not yet participated in gas exchange. The SpO₂ measurement thus measures oxygenation of arterial blood in the fingertip or other tissue being measured, whether that arterial blood is in arteries, capillaries, or is in both blood vessel types.

It is recognized herein that medical professionals often have a tendency to rely primarily on the SpO₂ vital sign to the exclusion of capnography data. This is due both to greater familiarity of many clinicians with SpO₂ as compared with capnography, and the recognition by clinicians that a low SpO₂ level is a direct clinical measure of an urgent medical problem, namely that the patient is not being sufficiently oxygenated. By contrast, interpretation of the capnography data, such as the etCO₂, is more complex, and may be more difficult for some medical professionals.

However, it is recognized herein that capnography is complementary to SpO₂ monitoring because capnometry can serve as a leading indicator by detecting a respiration problem before it manifests as reduced SpO₂ level. The capnography measures a direct product of blood-gas exchange in the lungs; whereas, SpO₂ measures a lagging metric of this blood-gas exchange and provides a clinical warning only after an insufficiency in transfer of oxygen to blood in the lungs occurring over an extended period of time produces a cumulative reduction in blood oxygenation.

Another way that capnography can be complementary to SpO₂ monitoring is in the case of a patient who is receiving supplemental oxygen. Here, the supplemental oxygen facilitates a high SpO₂ level, but in so doing may mask an underlying blood-gas exchange problem in the lungs or respiration rate and/or volume is low. Capnography, by directly measuring the CO₂ product of this blood-gas exchange in the lungs, can detect respiratory problems that may be masked in the SpO₂ measurement by the additional oxygenation provided by the supplemental oxygen.

In approaches disclosed herein, SpO₂ and capnography are synergistically combined to provide patient monitoring that more rapidly detects respiratory problems, and can detect respiratory problems that may otherwise be masked by supplemental oxygen, while still providing life-critical blood oxygenation monitoring via SpO₂ monitoring. In some embodiments, the disclosed approaches further provide synergistic clinical decision support. The SpO₂ and capnography information are analyzed separately to identify one or more clinical warnings, and these warnings are displayed in a ranked fashion based on urgency.

The RWI, by itself, does not consider blood oxygenation (or, more generally, the cardiac condition of the patient). In the following illustrative embodiments, the arterial oxygen saturation level (SpO₂) of the patient is combined with the RWI to calculate an index of the overall patient safety, referred to herein as a patient safety index (PSI). The illustrative PSI is a value in the range of 1 to 10, where 1 is the lowest score (patient needs immediate attention) and 10 is the highest score (healthy ventilation and oxygenation). It is possible for a patient to simultaneously have both inadequate oxygen saturation in the blood, indicated by low hemoglobin oxygen saturation, and adequate respiration, indicated by normal respiration rate and end-tidal CO₂ concentration.

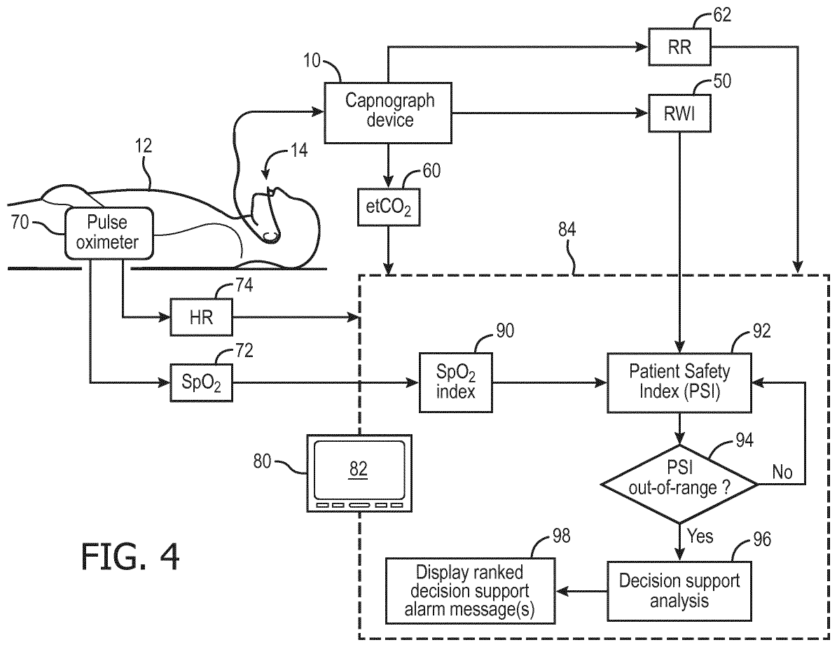

With reference to FIG. 4, an illustrative embodiment generating the PSI by combining the RWI and SpO₂ level is diagrammatically shown. The patient 12, patient accessory 14 (nasal cannula in this instance), and capnograph device 10 are as already described for the embodiment of FIG. 1. The capnograph device 10 outputs the respiratory well-being index (RWI) 50, an end-tidal carbon dioxide (etCO₂) value 60 and a respiratory rate (RR) value 62 determined from the capnogram signal 40, as also previously described with reference to FIG. 1. The illustrative embodiment of FIG. 4 does not output the capnogram signal waveform, the capnogram histogram, or the PQI (parameter quality index) values of the embodiment of FIG. 1, although any of these could also be outputs in variants of the embodiment of FIG. 4 if desired.

The illustrative embodiment of FIG. 4 further includes or has access to a pulse oximeter 70, which may for example be a fingertip pulse oximeter or the like. In a typical pulse oximeter design, light emitting diodes (LEDs) or other light sources transmit red light and infrared light through tissue (e.g. a fingertip) of the patient, and the transmission in these wavelengths is measured. As is known in the art, differential absorption at these different spectral locations enables extraction of arterial blood oxygen saturation (SpO₂) 72. Heart rate (HR) 74 may also be output by the pulse oximeter 70, obtained from fluctuations in the optical signals as blood volume in the monitored tissue (e.g. fingertip) fluctuates cyclically with each successive heartbeat. (The heart rate may additionally or alternatively be obtained from another sensor, e.g. an electrocardiogram or so forth).

A multi-parameter patient monitor 80 receives as inputs the RWI 50 and the etCO₂ value 60, and also optionally receives other physiological parameters such as RR 62 from the capnograph device 10, HR 74 from the pulse oximeter 70, blood pressure from a blood pressure monitor (components not shown), and/or so forth. The illustrative patient monitor 80 includes a display 82 and an electronic processor 84. As is conventional in patient monitoring, the electronic processor 84 is optionally programmed to display one or more of the received physiological parameters 60, 62, 72, 74 on the display 82, e.g. as a trend line and/or as numerical values, optionally averaged over an averaging time window. Physically, the patient monitor 80 may be variously embodied, e.g. as a bedside patient monitor, a nurses' station monitor, a wearable patient monitoring device, or so forth. Some illustrative examples of patient monitors include the various IntelliVue™ patient monitors available from Koninklijke Philips N.V., Eindhoven, the Netherlands. In other embodiments, the patient monitor 80 may be integrated with some other medical device—for example, the patient monitor 80 may be a component of a mechanical ventilator (not shown).

The electronic processor 84 of the illustrative patient monitor 80 of FIG. 4 is further programmed to compute the Patient Safety Index (PSI) as diagrammatically shown in FIG. 4. To this end, the SpO₂ value 72 is converted to an SpO₂ score or index 90, and the SpO₂ index 90 and the respiratory well-being index (RWI) 50 are combined to generate a patient safety index (PSI) 92 which may be utilized in various ways. In the illustrative example of FIG. 4, the PSI 92 is used as input to a decision operation 94 to detect a clinical problem. If the decision 94 is that a clinical problem is evidenced by the value of the PSI 92, then a decision support analysis 96 is triggered to analyze the SpO₂ and capnography data to identify alarm conditions such as low SpO₂ level, possible incorrect endotracheal tube placement, hypercarbia (i.e. abnormally elevated CO₂ in blood), or so forth. In an operation 98, any such alarm conditions are displayed on the display 82 of the patient monitor 80, e.g. as a list ranked by urgency (which in some embodiments may be a top-N list where N is a sub-set of the one, two, three, or more most urgent warnings).

In the following, an illustrative example of one suitable formulation of the PSI 92 is set forth.

In the illustrative example of the SpO₂ index 90, the arterial blood oxygen saturation (SpO₂) measurement 72 is input into a scoring function which outputs a score between +10 and −10. If the arterial blood oxygen saturation 72 is above an upper threshold (e.g. 94%), then the scoring function outputs the maximum scoring value of 10. For lower values of the oxygen saturation 72, the score decreases. If the arterial blood oxygen saturation 72 is below a lower threshold (e.g. 80%), then the scoring function outputs the minimum scoring value of −10.

In the illustrative example of calculating the PSI 92, a weighting factor is applied to the oxygen saturation score 90 and to the calculated RWI 50 from the capnography device 10. The weighted sum of these scores is the resulting PSI value. For example if the arterial blood oxygen saturation is 92% then the corresponding score may be 3. If the corresponding RWI is 5 and if the weights for both inputs is 0.5, the output PSI is 4, indicating that the patient may potentially be at risk. In the illustrative example, the choice of a scale in the range [−10,10] for the SpO₂ score ensures that a low SpO₂ value will draw down the combined PSI to ensure that it captures the clinically urgent situation in which the patient's blood oxygenation is low.

A variant embodiment of the arterial blood oxygen saturation scoring adjusts the SpO₂ score 90 for the SpO₂ when the patient is receiving supplemental oxygen. This adjustment captures the clinical reality that an arterial blood oxygen saturation that would be considered near normal (i.e. 94%) when the patient is breathing air would be considered low if the same patient is receiving supplemental oxygen through a nasal cannula, mask or endotracheal tube. To account for this difference in expected normal range, function generating the SpO₂ index 90 is shifted to lower value by a small amount (i.e. 2%) when it is known that the patient is receiving supplemental oxygen. This variant embodiment allows the PSI 92 to be more sensitive to low oxygen saturation values when the patient is receiving supplemental oxygen and saturation values are expected to be a little higher.

The determination that the patient is on supplemental oxygen may be based on a user input to the patient monitor 80 (e.g., when setting up the patient profile the nurse or other medical professional may select a radial input button indicating the patient is on supplemental oxygen). Alternatively, an automated mechanism for detecting that the patient is on supplemental oxygen may be utilized—for example, if the patient monitor 80 is integral with a mechanical ventilator or is connected to receive data from a mechanical ventilator, and the available data include fraction of inspired oxygen (FiO₂), then the patient monitor 80 may automatically detect whether the patient is on supplemental oxygen based on the FiO₂ value. In such embodiments, it is further contemplated to adjust the aforementioned small shift to lower value of the SpO₂ index 90 based on the supplemental oxygen level, e.g. a larger downward shift in the index value may be applied for higher FiO₂ value (as a higher fraction of inspired oxygen indicates more supplemental oxygen).

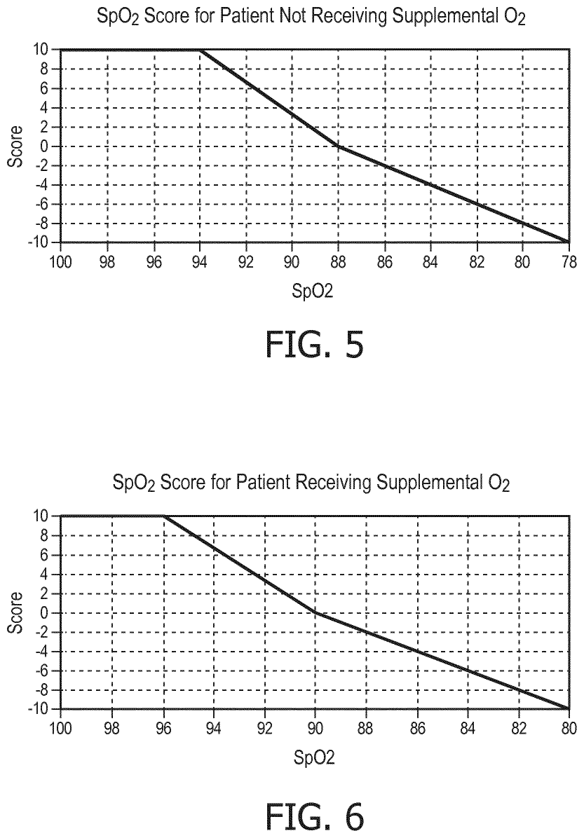

With reference to FIGS. 5 and 6, illustrative examples of the SpO₂ index-versus-SpO₂ value function suitably used to compute the SpO₂ index 90 are shown for the case of no supplemental oxygen (FIG. 5) and for the case of supplemental oxygen (FIG. 6). As seen in FIG. 5, with no supplemental oxygen the SpO₂ index score remains at its maximum value of 10 for SpO₂ values down to 94% (i.e., the upper threshold is 94%). As seen in FIG. 6, with supplemental oxygen the SpO₂ index score remains at its maximum value of 10 for SpO₂ values down to only 96% (i.e., the upper threshold is increased to 96%), reflecting that, for example, a 95% SpO₂ is usually deemed to be clinically acceptable for a patient without supplemental oxygen, but may be deemed to be abnormally low for a patient on supplemental oxygen. More generally, in some preferred embodiments the SpO₂ index 90 is computed using a monotonic function that has a minimum value (e.g. −10 in the illustrative examples) for values of SpO₂ at or below a lower threshold SpO₂ value (78% for the no supplemental oxygen scoring function of FIG. 5, or 80% for the supplemental oxygen scoring function of FIG. 6) and increases monotonically to a maximum value (e.g. +10 in the illustrative examples) for values of SpO₂ at or above an upper threshold SpO₂ value (94% for the no supplemental oxygen scoring function of FIG. 5, or 96% for the supplemental oxygen scoring function of FIG. 6).

In combining the SpO₂ index 90 and the RWI 50 to generate the PSI 92, the RWI and the SpO₂ values should reflect physiologic conditions corresponding to the same point in time. If these two input signals are misaligned in time, they may not work in concert to indicate patient safety. Because the RWI and the SpO₂ are derived from different physiologic signals, one measured by the capnography device 10 and the other by the pulse oximeter 70, there is a possibility that one may reflect an event or conditions that occurred before or after the other. In other words, the data streams from the two different devices 10, 70 may not be synchronized in time. Another cause of misalignment may be signal averaging. It may be beneficial to average the input signals to improve variability of the inputs. However, signal averaging delays the response of the signals, so that one or the other of the two signals may be delayed relative to the other signal (SpO₂ or capnometry). Various approaches can be used to synchronize the SpO₂ and capnography signals, e.g. using a common clock signal output to the two devices 10, 70 from the patient monitor 80, transmitting a synchronizing clock signal from one of the two devices 10, 70 to the other, or so forth. In another approach, an identifiable landmark in the signals can be used, for example if the capnography device 10 is a multifunction patient monitoring device that also measures heart rate then this heart rate may be used to synchronize with the HR 74 measured by the pulse oximeter 70 to synchronize the signals from the two devices 10, 70. These are merely illustrative synchronization approaches.

With reference to FIG. 4, some illustrative examples of embodiments of the decision support analysis 96 and decision support alarm messaging 98 are next described.

The PSI 92 may be displayed, e.g. as another patient data stream on the patient monitor 80. However, in the illustrative example of FIG. 4, the PSI 92 is not usually displayed, and in some embodiments is never displayed. Rather, the PSI 92 primarily serves as input to the decision 94 in order to detect a possible situation calling for clinical intervention. At the operation 94, if the calculated PSI 92 is below a threshold then a message is displayed. However, simply displaying a warning such as “PSI below safe threshold” is not especially informative to the nurse, doctor, or other clinician. Rather, in the illustrative embodiment of FIG. 4 the low PSI triggers the decision support analysis 96 which provides one or more clinically informative warning messages that are displayed in the alarm messaging operation 98. These messages are selected, and optionally displayed in ranked fashion, based on the impact that each input (RWI or SpO₂ score) has on the calculated PSI 92. The impact is the product of the imperfection in the score (10—score) and the weighting factor applied to the input. For example, if the SpO₂ index 90 is 3 and the weighting factor is 0.5, then the impact of the SpO₂ score on the PSI would be 1.5. If the SpO₂ has the higher impact on the PSI, then a message indication “low SpO₂” (or some other semantically similar message such as “Inadequate Oxygenation” or “Check Supplemental O₂”) would be displayed. If on the other hand, the RWI has the higher impact on the score, then a message based on the RWI would be shown.

An illustrative monitoring process performed using the embodiment of FIG. 4 is described in the following. For a given breath or period of time, calculate the RWI 50 from the capnography signal measured by the capnography device 10 as described previously herein with reference to FIG. 1. The SpO₂ measurement 72 corresponding in time to the breaths when the RWI was calculated are received, e.g. from the illustrative pulse oximeter 70. Based on the presence or absence of supplemental oxygen, select the correct score mapping function for SpO₂ (e.g. the scoring function of FIG. 5 for no supplemental oxygen, or the scoring function of FIG. 6 for supplemental oxygen. The SpO₂ value is mapped to the SpO₂ score or index 90 of FIG. 4. The PSI index value 92 is calculated as a weighted sum of the SpO₂ index score and the RWI value. At the decision 94, if the PSI index value 92 is less than a threshold value then the decision support analysis 96 is initiated. In one illustrative approach, a fault score is calculated for each of the inputs to the PSI 92 (that is, for each of the SpO₂ index score 90 and RWI 50). The fault score is suitably calculated as the product of the weighting factor and 10.0 minus the feature value. It is then determined which input (SpO₂ or RWI) has the highest fault score. If the SpO₂ has the higher fault score, then in the operation 98 a message is displayed indicating a low SpO₂. If the RWI has the higher fault score, then in the operation 98 a message is displayed based on the RWI. This latter output optionally is generated by performing further decision support analysis on the capnography data, e.g. as described herein with reference to FIG. 1.

Optionally, the PSI signal may be averaged over an extended time or over a number of breaths. For example, if the PSI is calculated every 5 seconds, it may be beneficial to display the average PSI calculated during the prior minute rather than display the resulting PSI as calculated every 5 seconds. This can help to avoid producing false alarms due to noise in the PSI data stream.

The illustrative example of FIG. 4 employs the multi-parameter patient monitor 80 as the host computation/display device for performing the operations that integrate capnography and SpO₂ data for improved patient monitoring. Implementing this processing at the patient monitor 80 is advantageous as such a multi-parameter patient monitor is a common “hub” where capnography data and SpO₂ data are collected. In the illustrative example the RWI 50 is further computed by processing performed by the capnograph device 10 as described with reference to FIG. 1. However, more generally, these various processing may be otherwise distributed over available electronic processing and display devices. For example, in another contemplated embodiment all processing is performed at the capnograph device, with the SpO₂ being an input to the capnograph device. In this arrangement the patient monitor may optionally be omitted. In another contemplated embodiment the capnograph device outputs the raw capnogram waveform to a patient monitoring device which performs both the RWI computation and the subsequent operations integrating the RWI and SpO₂. This approach enables the patient monitor to provide PSI-based monitoring in conjunction with any capnograph device that can output the raw capnogram. It will be still further appreciated that the operations that integrate capnography and SpO₂ data for improved patient monitoring may be embodied by a non-transitory storage medium storing instructions that are readable and executable by a microprocessor, microcontroller, or other electronic processor to perform the disclosed processing. Such non-transitory storage media may, by way of non-limiting illustration, include a hard disk drive or other magnetic storage medium, a flash memory, read-only memory (ROM) or other electronic storage medium, an optical disk or other optical storage medium, various combinations thereof, or so forth.

As further contemplated variants, the disclosed RWI is to be understood to be a non-limiting illustrative example of a capnography index which represents the patient's well-being as indicated by the capnogram measured by the capnograph device 10. More generally, other of capnography index formulations may be employed. As another illustrative example, the end-tidal CO₂ (etCO₂) may be used as the capnography index, optionally scaled between minimum and maximum index values similarly to the disclosed scaling operation for SpO₂ (e.g. the illustrative examples of FIGS. 5 and 6). It should be noted that the capnography index may be computed using any information derived from the capnogram, e.g. the illustrative RWI is computed based on the carbon dioxide concentration or partial pressure and also respiratory rate (RR) information derived from the capnogram.

The approach of FIG. 4 and variants thereof in combining the capnography and arterial blood oxygen saturation data reduces uncertainty and confusion associated with capnometry and arterial blood oxygen saturation monitoring and allows clinicians with less expertise in interpreting capnography data to more effectively integrate capnography and SpO₂ monitoring into interpretation of patient monitoring. The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A patient monitoring device comprising:
a capnograph device configured to measure a capnogram for use in monitoring patient respiratory gases, wherein the capnogram comprises a computed histogram of carbon dioxide (CO₂) value counts versus binned CO₂ level acquired over a given time interval that encompasses a number of patient breaths, wherein the computed histogram is periodically updated, the capnograph device further configured to (i) determine an end-tidal carbon dioxide (etCO₂) from the capnogram, (ii) compute an etCO₂ parameter quality index (etCO₂ PQI) using one or more quantitative capnogram waveform metrics computed from the capnogram, (iii) determine a respiration rate (RR) value from the capnogram, and (iv) compute a RR parameter quality index (RR PQI) using both (iv)(a) the RR value and (iv)(b) the etCO₂ PQI;
a pulse oximeter configured to measure an arterial blood oxygen saturation (SpO₂) of the patient; and
an electronic processor programmed to:
generate a capnography index indicative of patient well-being from the capnogram measured by the capnograph device, wherein the capnography index is computed based in part on (i) the etCO₂ PQI and (ii) the RR PQI;
generate an arterial blood oxygen saturation (SpO₂) index indicative of patient well-being from the SpO₂ measured by the pulse oximeter;
compute a patient safety index (PSI) from the capnography index and the SpO₂ index, wherein the capnography index is re-generated, and the etCO₂ PQI, and RR PQI are re-calculated, each time that the capnograph device updates the computed histogram; and
compute one or more clinical warnings determined based at least in part on the patient safety index; and
a display component configured to display at least one of the computed one or more clinical warnings, wherein the electronic processor is further programmed (i) to output the computed one or more clinical warnings to the display component in response to the patient safety index being below a PSI threshold value, and (ii) to suppress the output of the computed one or more clinical warnings to the display component in response to the patient safety index being below the PSI threshold value and the capnography index, etCO₂PQI or RR PQI being lower than a given index threshold value.

2. The patient monitoring device of claim 1 wherein the capnography index is further generated from information derived from the capnogram including at least a concentration or partial pressure of carbon dioxide and respiratory rate information.

3. The patient monitoring device of claim 1 wherein the SpO₂ index is generated using a monotonic function that has a minimum value for values of SpO₂ at or below a lower threshold SpO₂ value and that increases monotonically to a maximum value for values of SpO₂ at or above an upper threshold SpO₂ value.

4. The patient monitoring device of claim 3 wherein the minimum value of the monotonic function is less than zero and the maximum value of the monotonic function is greater than zero.

5. The patient monitoring device of claim 3 wherein the SpO₂ index is generated using the monotonic function with higher values of the lower threshold SpO₂ value and the upper threshold SpO₂ value when the SpO₂ is measured with supplemental oxygen and with lower values of the lower threshold SpO₂ value and the upper threshold SpO₂ value when the SpO₂ is measured without supplemental oxygen.

6. The patient monitoring device of claim 5 wherein the electronic processor is further programmed to identify whether supplemental oxygen is in use based on a received fraction of inspired oxygen (FiO₂).

7. The patient monitoring device of claim 1 wherein the patient safety index is computed as a weighted sum of the capnography index and the SpO₂ index.

8. The patient monitoring device of claim 1 wherein the electronic processor is further programmed to threshold the patient safety index and to compute the one or more clinical warnings conditional on the thresholding.

9. The patient monitoring device of claim 1 wherein the one or more clinical warnings are computed by operations including:
determining whether the capnography index or the SpO₂ index indicates a more urgent clinical warning by comparing a component of the patient safety index computed from the capnography index and a component of the patient safety index computed from the SpO₂ index;
computing the clinical warning using the capnogram if the capnography index indicates a more urgent clinical warning than the SpO₂ index; and
computing the clinical warning using the SpO₂ if the SpO₂ index indicates a more urgent clinical warning than the capnography index.

10. The patient monitoring device of claim 1 comprising a multi-parameter patient monitor including the display and the electronic processor.

11. The patient monitoring device of claim 10 wherein the electronic processor further includes an electronic processor of the capnometer device, and at least the capnography index is computed by the electronic processor of the capnometer device.

12. A non-transitory storage medium storing instructions readable and executable by an electronic processor to perform patient monitoring comprising:
generating a capnography index indicative of patient well-being from a capnogram measured by a capnograph device, the capnogram for use in monitoring patient respiratory gases, wherein the capnogram comprises a computed histogram of carbon dioxide (CO₂) value counts versus binned CO₂ level acquired over a given time interval that encompasses a number of patient breaths, wherein the computed histogram is periodically updated, the capnograph device further configured to (i) determine an end-tidal carbon dioxide (etCO₂) from the capnogram, (ii) compute an etCO₂ parameter quality index (etCO₂ PQI) using one or more quantitative capnogram waveform metrics computed from the capnogram, (iii) determine a respiration rate (RR) value from the capnogram, and (iv) compute a RR parameter quality index (RR PQI) using both (iv)(a) the RR value and (iv)(b) the etCO₂ PQI, wherein generating the capnography index includes wherein the capnography index is computed based in part on (i) the etCO₂PQI and (ii) the RR PQI;
generating an arterial blood oxygen saturation (SpO₂) index of the patient indicative of patient well-being from SpO₂ measured by a pulse oximeter; and
computing a patient safety index (PSI) (92) from the capnography index and the SpO₂ index, wherein the capnography index is re-generated, and the etCO₂PQI, and RR PQI are re-calculated, each time that the capnograph device updates the computed histogram;
computing one or more clinical warnings determined based at least in part on the patient safety index; and
outputting the computed one or more clinical warnings to a display component configured to display at least one of the computed one or more clinical warnings in response to the patient safety index being below a PSI threshold value, and suppressing the outputting of the computed one or more clinical warnings to the display component in response to the patient safety index being below the PSI threshold value and the capnography index, etCO₂PQI or RR PQI being lower than a given index threshold value.

13. The non-transitory storage medium of claim 12 wherein the capnography index is further generated from information derived from the capnogram including at least a concentration or partial pressure of carbon dioxide and respiratory rate information.

14. The non-transitory storage medium of claim 12 wherein the SpO₂ index is generated using a monotonic function that has a minimum value for values of SpO₂ at or below a lower threshold SpO₂ value and that increases monotonically to a maximum value for values of SpO₂ at or above an upper threshold SpO₂ value.

15. The non-transitory storage medium of claim 14 wherein the minimum value of the monotonic function is less than zero and the maximum value of the monotonic function is greater than zero.

16. The non-transitory storage medium of claim 14 wherein the SpO₂ index is generated using the monotonic function with higher values of the lower threshold SpO₂ value and the upper threshold SpO₂ value when the SpO₂ is measured with supplemental oxygen is in use and with lower values of the lower threshold SpO₂ value and the upper threshold SpO₂ value when the SpO₂ is measured without supplemental oxygen in use.

17. The non-transitory storage medium of claim 16 wherein the performed patient monitoring further comprises: identifying whether supplemental oxygen is in use based on a received fraction of inspired oxygen (FiO₂).

18. The non-transitory storage medium of claim 12 wherein the patient safety index is computed as a weighted sum of the capnography index and the SpO₂ index.

19. The non-transitory storage medium of claim 12, wherein the one or more clinical warnings are computed by operations including: determining a more urgent component by comparing relative contribution to the patient safety index of the capnography index and the SpO₂ index, the clinical warning being computed using data of the more urgent component.

20. (canceled)

21. A patient monitoring method comprising:
measuring a capnogram using a capnograph device the capnogram for use in monitoring patient respiratory gases, wherein the capnogram comprises a computed histogram of carbon dioxide (CO₂) value counts versus binned CO₂ level acquired over a given time interval that encompasses a number of patient breaths, wherein the computed histogram is periodically updated, the capnograph device further configured to (i) determine an end-tidal carbon dioxide (etCO₂) from the capnogram, (ii) compute an etCO₂ parameter quality index (etCO₂ PQI) using one or more quantitative capnogram waveform metrics computed from the capnogram, (iii) determine a respiration rate (RR) value from the capnogram, and (iv) compute a RR parameter quality index (RR PQI) using both (iv)(a) the RR value and (iv)(b) the etCO₂ POI;
measuring arterial blood oxygen saturation (SpO₂) of the patient using a pulse oximeter; and
generating, via an electronic processor, a capnography index indicative of patient well-being from the capnogram, wherein the capnography index is computed based in part on (i) the etCO₂ PQI and (ii) the RR PQI, generating an SpO₂ index indicative of patient well-being from the SpO₂, and computing a patient safety index (PSI) from the capnography index and the SpO₂ index, wherein the capnography index is re-generated, and the etCO₂ PQI, and RR PQI are re-calculated, each time that the capnograph device updates the computed histogram;
computing, via the electronic processor, one or more clinical warnings determined based at least in part on the patient safety index; and
outputting, via the electronic processor, the computed one or more clinical warnings to a display component configured to display at least one of the computed one or more clinical warnings in response to the patient safety index being below a PSI threshold value, and suppressing the outputting of the computed one or more clinical warnings to the display component in response to the patient safety index being below the PSI threshold value and the capnography index, etCO₂ PQI or RR PQI being lower than a given index threshold value.

22. The patient monitoring method of claim 21 wherein: the capnography index is generated from information derived from the capnogram including at least a concentration or partial pressure of carbon dioxide and respiratory rate information; and the SpO₂ index is generated using a monotonic function that has a minimum value for values of SpO₂ at or below a lower threshold SpO₂ value and increases monotonically to a maximum value for values of SpO₂ at or above an upper threshold SpO₂ value.

23. The patient monitoring method of claim 22 wherein:
the minimum value of the monotonic function is less than zero and the maximum value of the monotonic function is greater than zero; and
the patient safety index is computed as a weighted sum of the capnography index and the SpO₂ index.

24. (canceled)
